(19) United States
(12) Patent Application Publication
(10) Pub. No.: US 2020/0034761 A1
(43) Pub. Date: Jan. 30, 2020

(54) SYSTEMS AND METHODS FOR AUTOMATICALLY GENERATING GENETIC RISK ASSESSMENTS

(71) Applicant: MYRIAD WOMEN'S HEALTH, INC., South San Francisco, CA (US)

(72) Inventors: Jonathan E. BROWN, Sausalito, CA (US); Matthew Joseph MAHER, Rescue, CA (US)

(73) Assignee: MYRIAD WOMEN'S HEALTH, INC., South San Francisco, CA (US)

(21) Appl. No.: 16/523,644

(22) Filed: Jul. 26, 2019

Related U.S. Application Data
(63) Continuation of application No. PCT/US2018/015333, filed on Jan. 25, 2018.
(60) Provisional application No. 62/452,467, filed on Jan. 31, 2017.
(60) Provisional application No. 62/531,297, filed on Jul. 11, 2017.
(60) Provisional application No. 62/567,786, filed on Oct. 4, 2017.

(51) Int. Cl. G06Q 10/06; G16H 10/20

(52) U.S. Cl. CPC G06Q 10/0635; G16H 10/60; G16H 10/20

(57) ABSTRACT

A computer-implemented method for automatically generating genetic risk assessments including (1) receiving a data share, derived from an electronic health records system, that indicates that a patient has scheduled an appointment with a healthcare provider, (2) in response to identifying that the patient has scheduled an appointment with the healthcare provider, sending a digital family history request to the patient in preparation for the scheduled appointment, the digital family history request including a link to a third-party portal and a request to access and complete an electronic family history form using the third-party portal, (3) receiving, through the third-party portal, responses to the family history form that the patient submitted using the third-party portal, (4) generating a genetic risk assessment for the patient based on the responses received through the third-party portal, and (5) based on the genetic risk assessment of (4), generating a three-generation patient pedigree.

### CROSS-REFERENCE TO RELATED APPLICATIONS

This application is a continuation of International Patent Application No. PCT/US2018/015333, filed Jan. 25, 2018, which claims priority to U.S. Provisional Application No. 62/452,467, filed Jan. 31, 2017, and to U.S. Provisional Application No. 62/531,297, filed Jul. 11, 2017, and to U.S. Provisional Application No. 62/567,786, filed Oct. 4, 2017, the disclosure of each of which is incorporated herein by reference.

### FIELD OF THE INVENTION

following disclosure relates generally to systems and methods for automatically generating genetic risk assessments, and more particularly, to systems and methods for identifying at-risk individuals and then generating genetic risk assessments for multiple generations of those individuals' families.

### BACKGROUND

An estimated 16 million Americans meet DNA testing guidelines for inherited cancer. However, only a fraction of those are identified and referred to genetic testing centers. Identifying individuals who meet testing guidelines may be difficult for a variety of reasons: a lack of family history gathering, family history data that is ineffectively stored, a lack of information about genetic counseling, and/or genetic testing misconceptions (e.g., believed to be “too expensive”). Assessing genetic risk is a task that is traditionally left up to healthcare providers. However, assessing patients to effectively identify those who meet criteria may be difficult for healthcare providers due to the administrative burden of consistently capturing and assessing patient health history. The instant disclosure, therefore, identifies and addresses a need for improved systems and methods for automatically generating genetic risk assessments.

### SUMMARY

Disclosed herein is an improved computer-implemented method for automatically generating genetic risk assessments. The method includes (1) receiving a data share, derived from an electronic health records system, that indicates that a patient has scheduled an appointment with a healthcare provider, (2) in response to identifying that the patient has scheduled an appointment with the healthcare provider, sending a digital family history request to the patient in preparation for the scheduled appointment, the digital family history request including a link to a third-party portal and a request to access and complete an electronic family history form using the third-party portal, (3) receiving, through the third-party portal, responses to the family history form that the patient submitted using the third-party portal, (4) generating a genetic risk assessment for the patient based on the responses received through the third-party portal, and (5) based on the genetic risk assessment of (4), generating a three-generation patient pedigree.

Disclosed herein is a computer-implemented method for automatically generating genetic risk assessments, at least a portion of the method being performed by a computing device comprising at least one processor. The method comprises receiving a data share, derived from an electronic health records system, that indicates that a patient has scheduled an appointment with a healthcare provider. In response to identifying that the patient has scheduled an appointment with the healthcare provider, the method comprises sending a digital family history request to the patient in preparation for the scheduled appointment, the digital family history request comprising a link to a third-party portal and a request to access and complete an electronic family history form using the third-party portal. Next, the method comprises receiving, through the third-party portal, responses to the family history form that the patient submitted using the third-party portal. Then, the method comprises generating a genetic risk assessment for the patient based on the responses received through the third-party portal. The method also comprises generating, based at least in part on the genetic risk assessments, a three-generation patient pedigree based on the number of aunts/uncles, number of brothers/sisters, and the number of son/daughters of the patient.

### BRIEF DESCRIPTION OF THE DRAWINGS

The accompanying drawings illustrate a number of example embodiments and are a part of the specification. Together with the following description, these drawings demonstrate and explain various principles of the instant disclosure.

FIG. 1 is a block diagram of an example system for automatically generating genetic risk assessments, in accordance with one embodiment of the present disclosure.

FIG. 2 is a block diagram of an additional example system for automatically generating genetic risk assessments, in accordance with one embodiment of the present disclosure.

FIG. 3 is a flow diagram of an example method for automatically generating genetic risk assessments, in accordance with one embodiment of the present disclosure.

FIG. 4 is a block diagram of example introduction, background, and family history sections of an exemplary electronic family history form, in accordance with one embodiment of the present disclosure.

FIG. 5 is a block diagram of an example results section with example genetic risk assessment, educational information, and testing subsections of an electronic family history, in accordance with one embodiment of the present disclosure.

FIG. 6 is a block diagram of an example patient report, in accordance with one embodiment of the present disclosure.

FIG. 6A is a block diagram of an example three-generation patient report, in accordance with one embodiment of the present disclosure.

FIG. 7 is a block diagram of an example aggregate report, in accordance with one embodiment of the present disclosure.

FIG. 8 is a block diagram of an example computing system capable of implementing one or more of the embodiments described and/or illustrated herein, in accordance with one embodiment of the present disclosure.

FIG. 9 is a block diagram of an example computing network capable of implementing one or more of the embodiments described and/or illustrated herein, in accordance with one embodiment of the present disclosure.

Throughout the drawings, identical reference characters and descriptions indicate similar, but not necessarily identical, elements. While the example embodiments described herein are susceptible to various modifications and alternative forms, specific embodiments have been shown by way of example in the drawings and will be described in detail herein. However, the example embodiments described herein are not intended to be limited to the particular forms disclosed. Rather, the instant disclosure covers all modifications, equivalents, and alternatives falling within the scope of the appended claims.

### DETAILED DESCRIPTION

The present disclosure is generally directed to systems and methods for automatically generating genetic risk assessments. As will be described in greater detail below, the disclosed systems and methods may enable automatically screening a clinic's patients to identify patients who are at risk for inherited cancer and/or who qualify for genetic testing. In some examples, the disclosed systems and methods include (i) automatically receiving data shares (e.g., from the electronic health records system of a clinic) including a list of patients with upcoming appointments at the clinic and (ii) automatically sending such patients a digital link to fill out an electronic family history form using a third-party portal. By automatically sending patients requests to digitally fill out an electronic family history form (e.g., based on data retrieved from data shares), the disclosed systems and methods may increase the number of patients from whom genetic screening information is collected. Digitally obtaining such information may also enable storing the data in a structured data format (as opposed to storing a paper copy filled out at a clinic and/or a photocopied version of such a paper copy), thereby improving the trackability and usefulness of the collected data.

In some examples, the disclosed systems and methods may enable analyzing the responses provided by the patients to generate risk assessments. In one example, each patient may be provided with his or her risk assessment information. Additionally or alternatively, the generated risk assessments may be provided to the patient's healthcare provider. In one embodiment, generated risk assessments for multiple patients may be aggregated into an aggregate report for the healthcare provider.

In some examples, the disclosed systems and methods may use the collected genetic risk assessments to screen a clinic's patients for patients who meet DNA testing guidelines for inherited cancer. In these examples, the disclosed systems and methods may also enable automatically providing the clinic with a list of patients who meet DNA testing guidelines for inherited cancer and/or with whom members of the clinic should discuss genetic testing and/or counseling. In some examples, this information may be collected and/or compiled based on the clinic's appointment schedule. For example, the disclosed systems and methods may screen patients who have upcoming appointments and provide the results to the clinic prior to the appointments (e.g., on a daily or weekly basis).

By automatically generating risk assessments for patients (e.g., using computer-implemented methods), the disclosed systems and methods may increase the number of patients who are identified as being at risk for inherited cancer and informed about genetic testing services. If a patient is identified as being at risk, the disclosed systems and methods may then capture family size in order to generate a multi-generation pedigree in order to increase the number of patients who may be identified as being at risk for inherited cancer and informed about genetic testing services. In addition, the systems and methods described herein may improve the functioning of electronic health records systems (e.g., by providing electronic health records systems with genetic screening information in a structured data format).

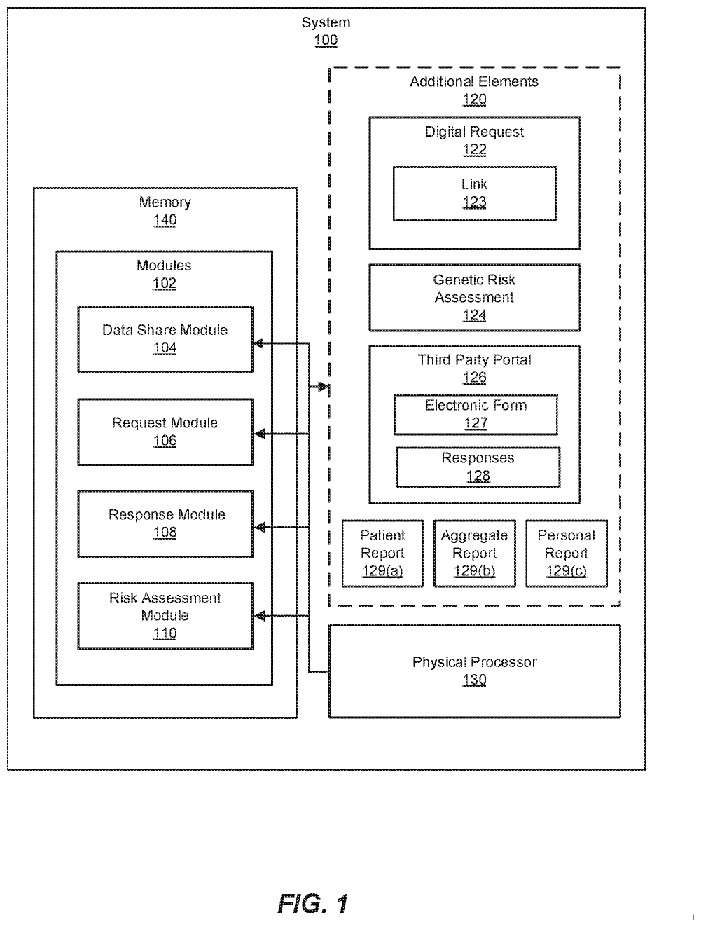

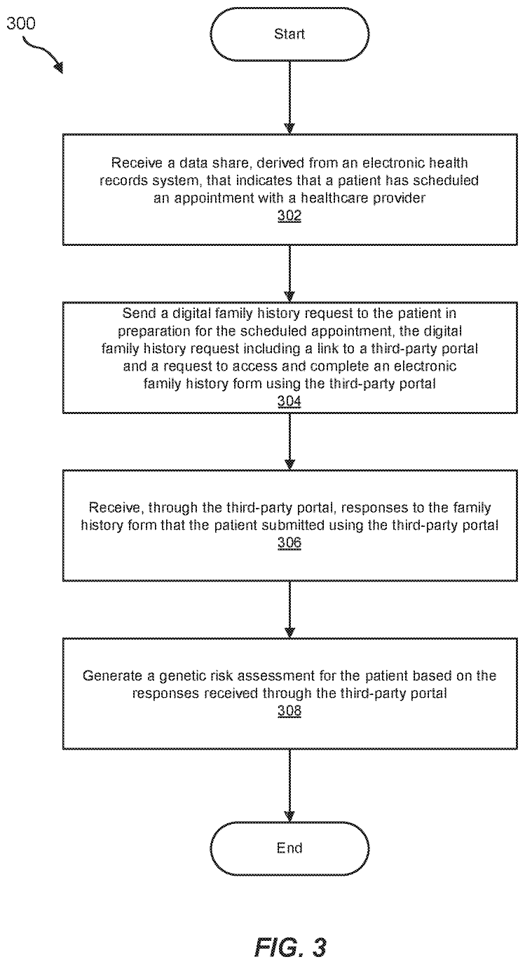

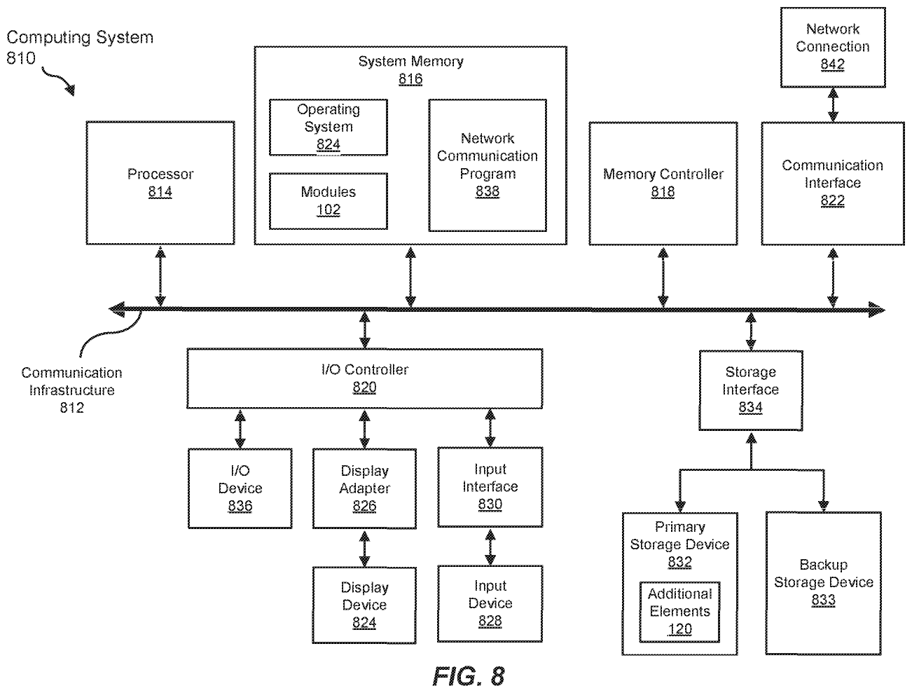

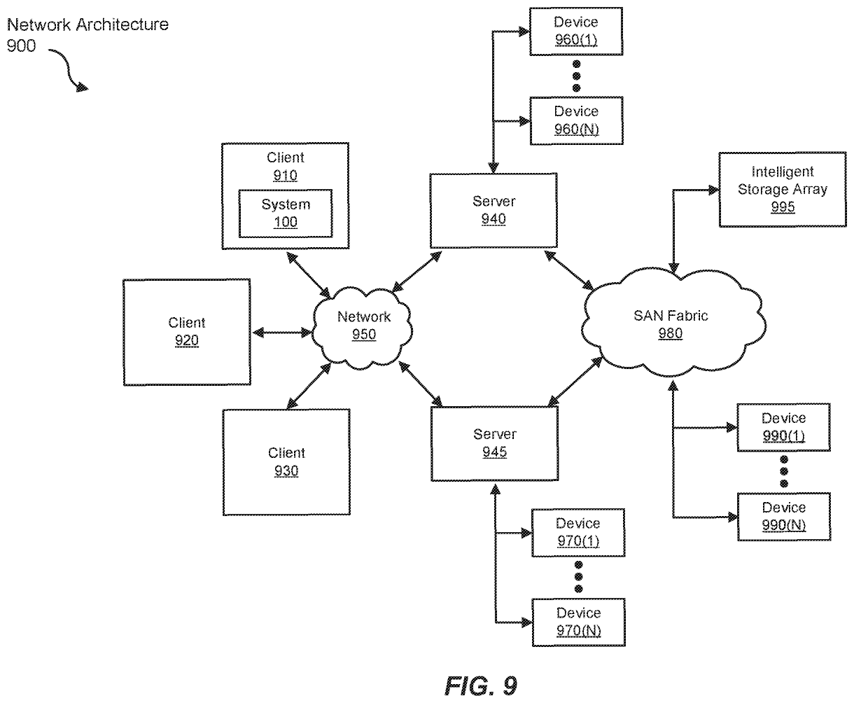

The following will provide, with reference to FIGS. 1-2 and 4-7, detailed descriptions of example systems for automatically generating genetic risk assessments. Detailed descriptions of corresponding computer-implemented methods will also be provided in connection with FIG. 3. In addition, detailed descriptions of an exemplary computing system and network architecture capable of implementing one or more of the embodiments described herein will be provided in connection with FIGS. 8 and 9, respectively.

FIG. 1 is a block diagram of exemplary system 100 for automatically generating genetic risk assessments. As illustrated in this figure, exemplary system 100 may include one or more modules 102 for performing one or more tasks. For example, and as will be explained in greater detail below, exemplary system 100 may include a data share module 104 that receives a data share, derived from an electronic health records system, that indicates that a patient has scheduled an appointment with a healthcare provider. Exemplary system 100 may additionally include a patient engagement module 106 that sends a digital family history request to the patient in preparation for the scheduled appointment, the digital family history request including a link to a third-party portal and a request to access and complete an electronic family history form using the third-party portal. Exemplary system 100 may also include a response module 108 that receives, through the third-party portal, responses to the family history form that the patient submitted using the third-party portal. Additionally, exemplary system 100 may include a risk assessment module 110 that generates a genetic risk assessment for the patient based on the responses received through the third-party portal. Although illustrated as separate elements, one or more of modules 102 in FIG. 1 may represent portions of a single module or application.

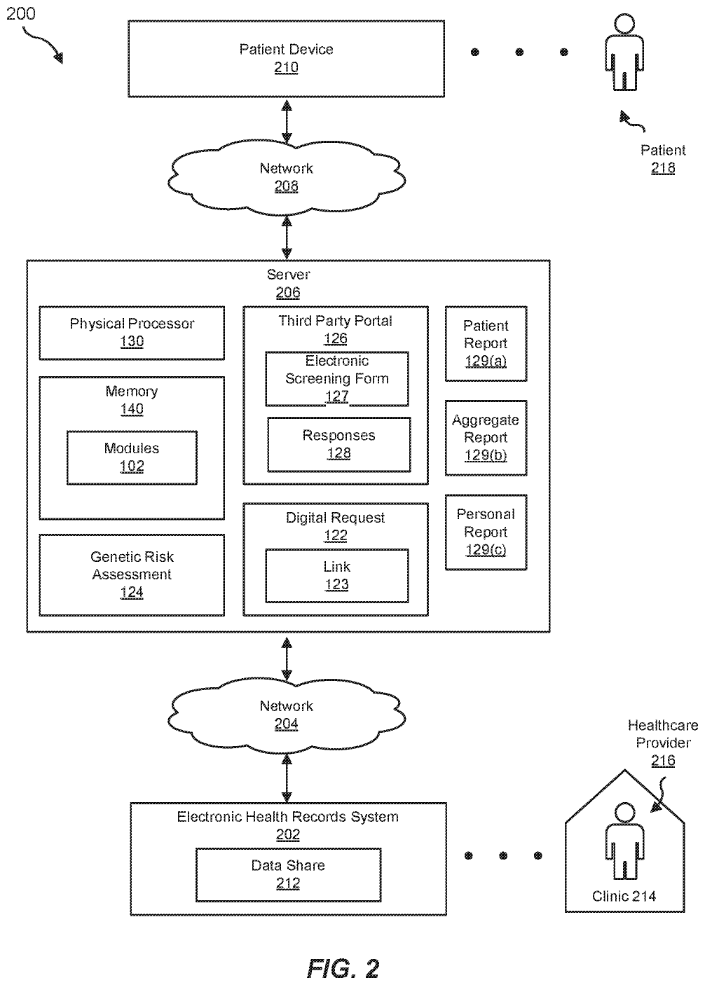

In certain embodiments, one or more of modules 102 in FIG. 1 may represent one or more software applications or programs that, when executed by a computing device, may cause the computing device to perform one or more tasks. For example, and as will be described in greater detail below, one or more of modules 102 may represent modules stored and configured to run on one or more computing devices, such as the devices illustrated in FIG. 2 (e.g., electronic health records system 202, server 206, and/or patient device 210). One or more of modules 102 in FIG. 1 may also represent all or portions of one or more special purpose computers configured to perform one or more tasks.

As illustrated in FIG. 1, example system 100 may also include one or more memory devices, such as memory 140. Memory 140 generally represents any type or form of volatile or non-volatile storage device or medium capable of storing data and/or computer readable instructions. In one example, memory 140 may store, load, and/or maintain one or more of modules 102. Examples of memory 140 include, without limitation, Random Access Memory (RAM), Read Only Memory (ROM), flash memory, Hard Disk Drives (HDDs), Solid-State Drives (SSDs), optical disk drives, caches, variations or combinations of one or more of the same, and/or any other suitable storage memory.

As illustrated in FIG. 1, example system 100 may also include one or more physical processors, such as physical processor 130. Physical processor 130 generally represents any type or form of hardware-implemented processing unit capable of interpreting and/or executing computer-readable instructions. In one example, physical processor 130 may access and/or modify one or more of modules 102 stored in memory 140. Additionally or alternatively, physical processor 130 may execute one or more of modules 102 to facilitate automatically generating genetic risk assessments. Examples of physical processor 130 include, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, and/or any other suitable physical processor.

As illustrated in FIG. 1, example system 100 may also include one or more additional elements 120, such as a digital request 122 (including a link 123), a genetic risk assessment 124, a third-party portal 126, an electronic screening form 127 (accessed via third party portal 126), responses 128 (collected via electronic screening form 127), a patient report 129(a), an aggregate report 129(b), and a personal report 129(c). Each of these additional elements will be discussed in greater detail below in connection with FIG. 3.

Example system 100 in FIG. 1 may be implemented in a variety of ways. For example, all or a portion of example system 100 may represent portions of example system 200 in FIG. 2. As shown in FIG. 2, system 200 may include an electronic health records system 202 in communication with a server 206 via a network 204 and a patient device 210 in connection with server 206 via a network 208. In one example, all or a portion of the functionality of modules 102 may be performed by electronic health records system 202, server 206, patient device 210 and/or any other suitable computing system.

As will be described in greater detail below, one or more of modules 102 from FIG. 1 may, when executed by at least one processor of electronic health records system 202, server 206, and/or patient device 210, enable electronic health records system 202, server 206, and/or patient device 210 to automatically generate genetic risk assessments. For example, and as will be described in greater detail below, data share module 104 may receive a data share 212, derived from electronic health records system 202, that indicates that a patient 218 has scheduled an appointment with a healthcare provider 216 and/or a clinic 214 associated with electronic health records system 202. In response to identifying that patient 218 has scheduled the appointment, patient engagement module 106 may send digital request 122 to patient device 210 in preparation for the scheduled appointment. Digital request 122 may include a link to third-party portal 126 and a request to access and complete electronic screening form 127 using third-party portal 126. Response module 108 may receive, through third-party portal 126, responses 128 to electronic screening form 127 that patient 218 submits using third-party portal 126. Then, risk assessment module 110 may generate genetic risk assessment 124 for patient 218 based on responses 128 received through third-party portal 126.

Electronic health records system 202 generally represents any type or form of computing device that is capable of maintaining a systematized collection of patient centered and/or population-centered records. Such records may include, without limitation, patient health information, patient appointment schedules, and/or patient medical and treatment histories (e.g., diagnoses, medications, current treatment plans, immunization dates, radiology images, laboratory results, etc.).

In some examples, electronic health records system 202 may automate and streamline healthcare provider workflow. In one embodiment, electronic health records system 202 may be managed by a healthcare provider and/or clinic. In this embodiment, electronic health records system 202 may be configured to store records in a digital format capable of being shared with other healthcare providers or organizations involved in a patient's care, such as laboratories, genetic screening specialists, etc. Additional examples of electronic health records system 202 may include, without limitation, web servers, storage servers, and/or database servers configured to run certain software applications and/or provide various web, storage, and/or database services. Although illustrated as a single entity in FIG. 2, electronic health records system 202 may include and/or represent a plurality of servers that work and/or operate in conjunction with one another.

Server 206 generally represents any type or form of computing device that is capable of automatically collecting and/or generating genetic risk assessments. In some examples, server 206 may be managed by an entity that provides genetic screening, counseling, and/or testing services. Additional examples of server 206 include, without limitation, web servers, storage servers, and/or database servers configured to run certain software applications and/or provide various web, storage, and/or database services. Although illustrated as a single entity in FIG. 2, server 206 may include and/or represent a plurality of servers that work and/or operate in conjunction with one another.

Network 204 and network 208 generally represent any medium or architecture capable of facilitating communication or data transfer. In one example, network 204 may facilitate communication between electronic health records system 202 and server 206. Additionally or alternatively, network 208 may facilitate communication between server 206 and patient device 210. In these examples, network 204 and/or network 208 may facilitate communication or data transfer using wireless and/or wired connections. Examples of network 204 and/or network 208 include, without limitation, an intranet, a Wide Area Network (WAN), a Local Area Network (LAN), a Personal Area Network (PAN), the Internet, Power Line Communications (PLC), a cellular network (e.g., a Global System for Mobile Communications (GSM) network), portions of one or more of the same, variations or combinations of one or more of the same, and/or any other suitable network.

Patient device 210 generally represents any type or form of device maintained by a patient of a healthcare provider (e.g., a patient with an upcoming appointment). Examples of patient device 210 include, without limitation, laptops, tablets, desktops, servers, cellular phones, Personal Digital Assistants (PDAs), multimedia players, embedded systems, wearable devices (e.g., smart watches, smart glasses, etc.), gaming consoles, variations or combinations of one or more of the same, and/or any other suitable computing device.

FIG. 3 is a flow diagram of an example computer-implemented method 300 for automatically generating genetic risk assessments. The steps shown in FIG. 3 may be performed by any suitable computer-executable code and/or computing system, including system 100 in FIG. 1, system 200 in FIG. 2, and/or variations or combinations of one or more of the same. In one example, each of the steps shown in FIG. 3 may represent an algorithm whose structure includes and/or is represented by multiple sub-steps, examples of which will be provided in greater detail below.

As illustrated in FIG. 3, at step 302, one or more of the systems described herein may receive a data share, derived from an electronic health records system, that indicates that a patient has scheduled an appointment with a healthcare provider. For example, data share module 104 may, as part of server 206 in FIG. 2, receive data share 212, derived from electronic health records system 202, that indicates that patient 218 has scheduled an appointment with healthcare provider 216.

As used herein, the term “data share” generally refers to any type or form of secure and/or encrypted data exchange between two servers (e.g., between electronic health records system 202 and server 206). In one example, data share 212 may represent a bulk patient data secure file transfer, derived from electronic health records system 202, to server 206. In some examples, the file transfer may include transferring a comma-separated values (CSV) file that includes the patient information. In one embodiment, the transfer may represent an SSL/HTTPS transfer using a web portal such as BOX.COM.

In one example, the information transferred in data share 212 may include a list of patients who are scheduled for appointments with healthcare provider 216 (or with clinic 214 associated with healthcare provider 216) during a specified time period. For example, data share 212 may include a list of all of the patients who have appointments at clinic 214 during the upcoming week.

Electronic health records system 202 may be configured to send data share 212 (e.g., automatically) in response to a variety of events. For example, electronic health records system 202 may be configured to send data share 212 in response to identifying that patient 218 has scheduled an appointment. Additionally or alternatively, electronic health records system 202 may be configured to automatically send data share 212 a designated number of days before and/or after the scheduled appointment. To give a specific example, electronic health records system 202 may be configured to send data share 212 one week before a scheduled appointment and/or on the Monday preceding a scheduled appointment.

In one embodiment, electronic health records system 202 may be configured to send data shares to data share module 104 on a periodic basis. To give a specific example, electronic health records system 202 may be configured to send a data share to data share module 104 once a week that includes a list of patients who have appointments scheduled for that week.

At step 304, one or more of the systems described herein may, in response to identifying that the patient has scheduled an appointment with the healthcare provider, send a digital family history request to the patient in preparation for the scheduled appointment, the digital family history request including a link to a third-party portal and a request to access and complete an electronic family history form using the third-party portal. For example, patient engagement module 106 may, as part of server 206 in FIG. 2, send digital request 122 to patient 218 in preparation for a scheduled appointment, digital request 122 including link 123 to third-party portal 126 and a request to access and complete an electronic screening form 127 using third-party portal 126. As another example, at step 304, one or more of the systems described herein may, in response to identifying that the patient has scheduled an appointment with the healthcare provider, send a resource submission request to the patient in preparation for the scheduled appointment, the resource submission request including a link to a third-party portal and a request to submit a resource information form using the third-party portal.

Patient engagement module 106 may send patient 218 a variety of types of digital requests. In some examples, patient engagement module 106 may send a text message request to a mobile device of patient 218, such as a smart phone, using a Short Message Service. Additionally or alternatively, patient engagement module 106 may send an email request to an email account of patient 218 using a Simple Mail Transfer Protocol (e.g., to be accessed by patient 218 via patient device 210).

In one example, patient engagement module 106 may send multiple digital requests successively (e.g., a first digital request at a first moment in time and a second digital request at a second moment in time). In some embodiments, patient engagement module 106 may send a subsequent digital request in response to determining that patient 218 did not respond as requested to a previous digital request. To give a specific example, patient engagement module 106 may send patient 218 a first digital request a week before his or her scheduled appointment. Then, if patient 218 does not respond to the first digital request as requested, patient engagement module 106 may send patient 218 a second digital request the day before the scheduled appointment. In one such example, the first digital request may represent a different type of request than the second digital request. For instance, the first digital request may represent an email message and the second digital request may represent a text message.

Digital request 122 may include a variety of content. In some examples, digital request 122 may include a request to access and complete electronic screening form 127 using third-party portal 126. Additionally, digital request 122 may include a link 123 to third-party portal 126. In one embodiment, digital request 122 may include content explaining a reason for the request. For example, digital request 122 may include the following text: “In preparation for your upcoming appointment with Dr. Holmes, please complete a family history form using the link provided below.”

As used herein, a third-party portal generally refers to any type or form of digital interface (such as a website) that provides a resource and/or service (such as an electronic family history form or a resource information form). In some examples, the third-party portal may be managed by an entity that provides genetic screening, counseling, and/or testing services. As used herein, an electronic family history form (e.g., electronic screening form 127) generally refers to any type or form of digital data structure, accessible via a third-party portal, that collects information (such as family history information) pertaining to screening patients for genetic testing and/or counseling. As used herein, a resource information form generally refers to any type or form of digital data structure, accessible via a third-party portal, that collects information pertaining to resource information of a given patient, such as insurance information, banking information, personal monetary information, or other financial information.

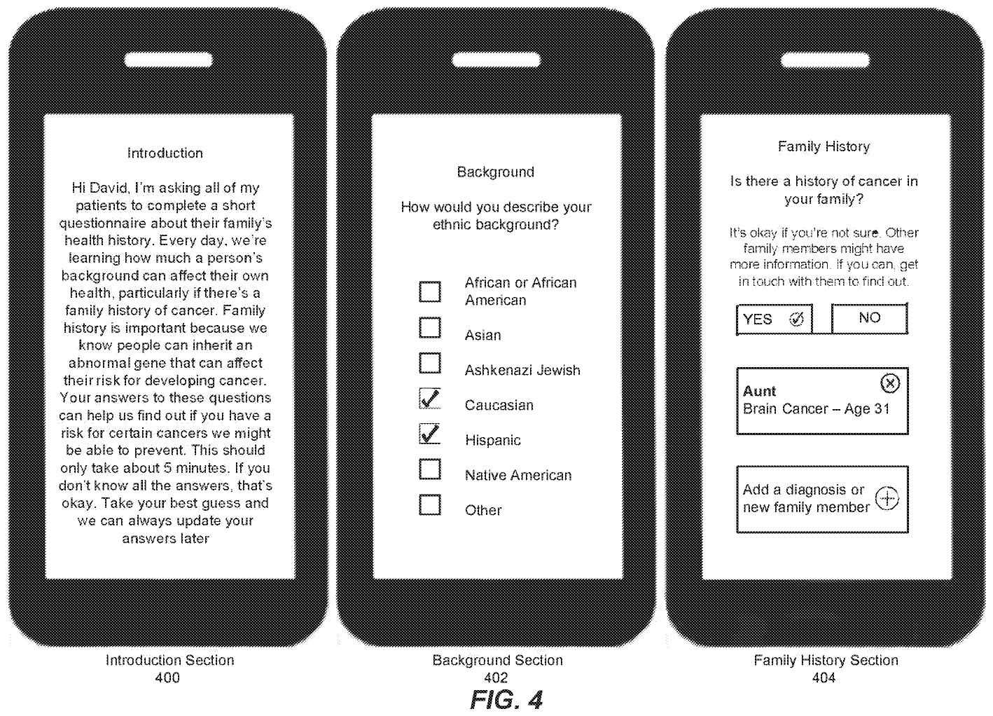

Electronic screening form 127 may include a variety of information. In some examples, electronic screening form 127 may include introductory information that explains the potential benefits of testing for genetic markers indicative of cancer. Using FIG. 4 as a specific example, electronic screening form 127 may include an introduction section 400 with the following text: “Hi David, I'm asking all of my patients to complete a short questionnaire about their family's health history. Every day, we're learning how much a person's background can affect their own health, particularly if there's a family history of cancer. Family history is important because we know people can inherit an abnormal gene that can affect their risk for developing cancer. Your answers to these questions can help us find out if you have a risk for certain cancers we might be able to prevent. This should only take about 5 minutes. If you don't know all the answers, that's okay. Take your best guess and we can always update your answers later.”

In some examples, electronic screening form 127 may include one or more questions relating to a patient's ethnic background. Returning to FIG. 4 as a specific example, electronic screening form 127 may include a background section 402 that asks, “How would you describe your ethnic background?” and provide selectable answers such as “African or African American, Asian, Ashkenazi Jewish, Caucasian, Hispanic, Native American, Other.”

In some examples, electronic screening form 127 may include one or more questions relating to the history of cancer in a patient's family and/or a patient's personal history of cancer. Returning to FIG. 4 as a specific example, electronic screening form 127 may include a family history section 404 that includes the question “Is there a history of cancer in your family?” and allows the patient to “Add a diagnosis or new family member.” The family history question may be accompanied with a prompt to reach out to family members, such as “It's okay if you're not sure. Other family members might have more information. If you can, get in touch with them to find out.” Family history section 404 may also allow a patient to digitally select an answer (e.g., “Yes” or “No”) and/or to digitally submit information (e.g., “Aunt—Brain Cancer—Age 31”). In some examples, electronic screening form 127 may include one or more questions relating to resource information for the patient, such as insurance information, banking information, personal monetary information, or other financial information.

In some examples, the electronic screening form 127 may also include one or more questions relating to patient's family size. Returning to FIG. 4 as a specific example, the electronic screening form 127 may include a family genealogy section that includes one or more questions, such as, “How many aunts/uncles?”, “How many brothers/sisters?”, and “How many sons/daughters?” The family size questions may be used to capture the patient's family size in order to generate a three-generation pedigree.

In one embodiment, the complexity of the questions in electronic screening form 127 may vary based on a patient's responses. For example, a response indicating one ethnicity may trigger different follow-up questions and/or a more extensive line of questions than a response indicating another ethnicity. Similarly, a response indicating one family history may trigger different follow-up questions and/or a more extensive line of questions than a response indicating a different family history. As another example, a response indicating a first set of insurance or financial information may trigger different follow-up questions and/or a more extensive line of questions than a response indicating a different set of insurance or financial information.

Patient engagement module 106 may send digital request 122 to patient 218 in response to a variety of determinations. In some examples, patient engagement module 106 may be configured to send an instance of digital request 122 to each patient indicated as having an upcoming appointment (or past appointment) in data share 212. In these examples, patient engagement module 106 may send digital request 122 to patient 218 in response to determining that patient 218 is indicated as having an upcoming appointment (or past appointment) in data share 212.

In other examples, patient engagement module 106 may be configured to send an instance of digital request 122 to a subset of the patients indicated as having an upcoming appointment (or past appointment) in data share 212. For example, patient engagement module 106 may be configured to (i) identify a subset of the patients indicated as having an upcoming appointment (or past appointment) in data share 212 who have already submitted responses to electronic screening form 127 (and/or who have otherwise already provided family history information) and (ii) send digital request 122 to patients who have not already submitted such responses and not send digital request 122 to patients who have already submitted such responses. In these examples, patient engagement module 106 may send digital request 122 to patient 218 in response to determining that patient 218 has not already provided family history information.

In one embodiment, patient engagement module 106 may further be configured to send patients who have already provided family history information or resource information (e.g., by submitting responses to electronic screening form 127) a modified version of digital request 122 that requests that a patient update the information previously provided (e.g., by sending a link to third-party portal 126 and a request to update the previously provided information using third-party portal 126). In these examples, patient engagement module 106 may send patient 218 the modified version of digital request 122 in response to determining that patient 218 has already provided family history information or resource information. In these examples, the disclosed systems and methods may receive the updated responses, generate an updated genetic risk assessment for the patient, and/or send updated information about the patient and/or a population that includes the patient to the healthcare provider and/or the clinic using the techniques described below in connection with step 306 and step 308.

Returning to FIG. 3, at step 306, one or more of the systems described herein may receive, through the third-party portal, responses to the family history form that the patient submitted using the third-party portal. For example, response module 108 may, as part of server 206 in FIG. 2, receive, through third-party portal 126, responses 128 to electronic screening form 127 that patient 218 submitted using third-party portal 126.

By collecting responses electronically (e.g., via third-party portal 126), the disclosed systems and methods may increase the time patients have to provide responses. This may allow patients to provide more thorough responses (e.g., by giving patients time to reach out to other family members for additional information) than would be afforded patients asked to provide family history in a waiting room. Also, by collecting responses electronically (as opposed to collecting via paper on a clipboard at a doctor's office), the disclosed systems and methods may obtain, and subsequently maintain, patient family history data or resource information in a searchable structure data format. This may provide patient data that is easy to analyze, search, and/or track.

At step 308, one or more of the systems described herein may generate a genetic risk assessment for the patient based on the responses received through the third-party portal. For example, risk assessment module 110 may, as part of server 206 in FIG. 2, generate genetic risk assessment 124 for patient 218 based on responses 128 received through third-party portal 126. As used herein, the term “risk assessment” generally refers to any type or form of assessment of a patient's risk level for having a genetic marker indicative of cancer. As another example, at step 308, one or more of the systems described herein may generate a resource assessment for the patient based on the responses received through the third-party portal. For example, a resource assessment module (not shown) may generate an assessment for patient 218 based on responses 128 received through third-party portal 126. As used herein, the term “assessment” may generally refer to any type or form of assessment of patient resources for having a specific test performed.

Risk assessment module 110 may use genetic risk assessment 124 in a variety of ways. In some examples, risk assessment module 110 may identify guidelines for testing for genetic markers indicative of cancer. In these examples, risk assessment module 110 may determine, based on genetic risk assessment 124, whether the guidelines recommend testing patient 218 for genetic markers indicative of cancer.

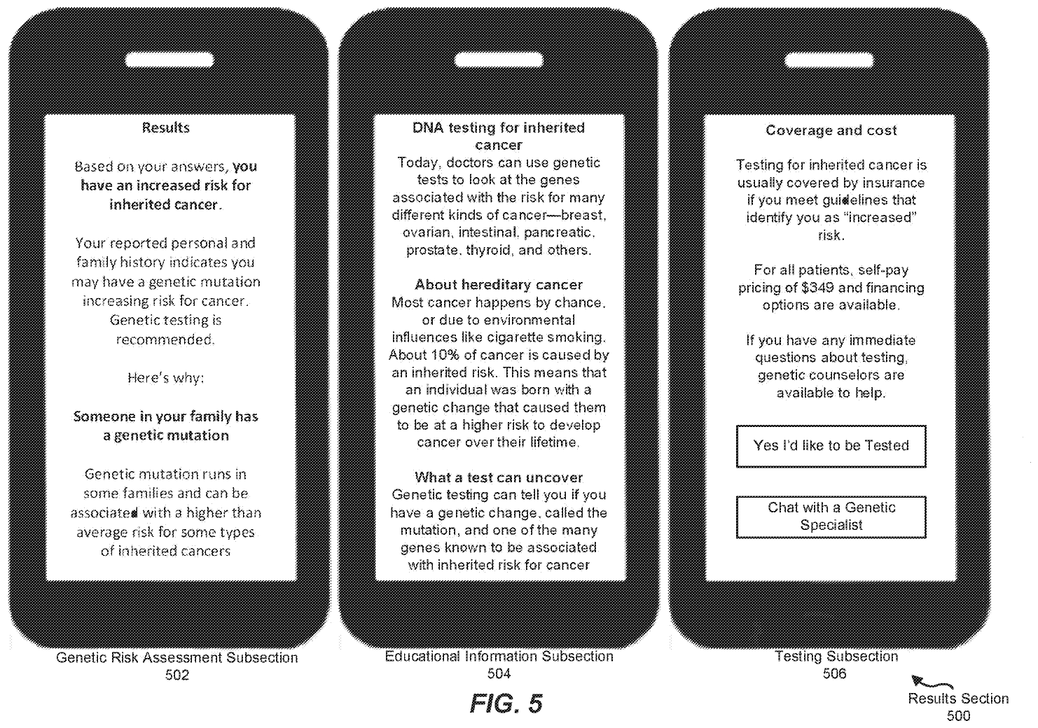

In some examples, risk assessment module 110 may use genetic risk assessment 124 to create a results section in electronic screening form 127 to present to patient 218 after he or she submits responses 128. Using FIG. 5 as a specific example, electronic screening form 127 may include a results section 500.

A results section of electronic screening form 127 may include a variety of information, such as a summary of genetic risk assessment 124, a reason for and/or explanation of genetic risk assessment 124, and/or a summary of responses 128 provided by patient 218. Using FIG. 5 as a specific example, results section 500 may include a genetic risk assessment subsection 502 that indicates the following: “Based on your answers, you have an increased risk for inherited cancer. Your reported personal and family history indicates you may have a genetic mutation increasing risk for cancer. Genetic testing is recommended. Here's why: Someone in your family has a genetic mutation. Genetic mutation runs in some families and can be associated with a higher than average risk for some types of inherited cancers.”

In certain embodiments, a results section of electronic screening form 127 may also include educational information (e.g., educational information that risk assessment module 110 identifies as pertinent to patient 218 based on genetic risk assessment 124). For example, a results section may include information describing inherited cancer and/or the importance of screening for indicators of an elevated genetic risk of inherited cancer. Using FIG. 5 as a specific example, an educational information subsection 504 within results section 500 may include a section entitled “DNA testing for inherited cancer” with information such as the following: “Today, doctors can use genetic tests to look at the genes associated with the risk for many different kinds of cancer—breast, ovarian, intestinal, pancreatic, prostate, thyroid, and others.” Also in this specific example, educational information subsection 504 may include a section entitled “About hereditary cancer” with information such as the following: “Most cancer happens by chance, or due to environmental influences like cigarette smoking. About 10% of cancer is caused by an inherited risk. This means that an individual was born with a genetic change that caused them to be at a higher risk to develop cancer over their lifetime.” Finally, in this specific example, educational information subsection 504 may include a section entitled “What a test can uncover” with information such as “Genetic testing can tell you if you have a genetic change, called a mutation, and one of the many genes known to be associated with inherited risk for cancer.”

As an alternative to inputting all family members first and asking health questions about each during the electronic screening form 127 of FIG. 4, the results section of electronic screening form 127 may streamline the process to capture the notable health history of family members and then asking about remaining family members without notable health history. Returning to FIG. 5 as a specific example, after learning about all notable health history in a family, in order to determine the size of the family without notable health history, the results section of electronic screening form 127 may include a family genealogy section that asks one or more questions, including “How many aunts/uncles?”, “How many brothers/sisters?”, and “How many sons/daughters?” If there is not any notable family health history, the patient simply answers the questions to determine the size of the family. The family size questions may be used to capture the patient's total family size to generate a three-generation pedigree and to determine the number of family members without notable health histories. By asking about those family members with notable health histories first, and then asking about the overall size of the family second, the user experience is simplified and more patient-friendly by limiting the questions for those family members that do not have a notable health history.

In some examples, a results section of electronic screening form 127 may also include information describing genetic testing (e.g., testing information that risk assessment module 110 identifies as pertinent to patient 218 based on genetic risk assessment 124). Testing information may include, for example, information describing a cost of testing for genetic markers indicative of cancer, criteria for health insurance coverage for such testing, and/or information for scheduling an appointment with a genetic testing center. In one example, testing information may include a prompt to discuss genetic testing with a healthcare provider during a patient's scheduled appointment or a link to contact a genetic specialist. Using FIG. 5 as a specific example, a testing subsection 506 may include a section entitled “Coverage and cost” with information such as the following: “Testing for inherited cancer is usually covered by insurance if you meet guidelines that identify you as ‘increased’ risk. For all patients, self-pay pricing of $349 and financing options are available. If you have any immediate questions about testing, genetic counselors are available to help.” Also, in this specific example, testing subsection 506 may allow the patient to select from various options such as “Yes, I'd like to be tested” and/or “Chat with a genetic specialist.”

In some examples, the information provided in a results section of electronic screening form 127 may vary based on the responses provided by a patient. For example, the results section may be more extensive if the responses indicate that the patient is at an increased risk for genetic markers indicative of cancer and less extensive if the responses indicate that the patient is at a low risk for genetic markers indicative of cancer.

In addition, or as an alternative, to creating a results section in electronic screening form 127, risk assessment module 110 may generate a personal report (i.e., personal report 129(c)) to provide to patient 218. Personal report 129(c) may include a variety of information including, for example, the patient's risk assessment, the patient's responses to the family history form, the patient's resource assessment, the patient's responses to the resource assessment, and/or educational information. In some examples, risk assessment module 110 may provide the patient with personal report 129(c) by sending a link to personal report 129(c) (e.g., using the same device used to send digital request 122 to the patient). For example, risk assessment module 110 may send a link to a mobile computing device such as a smart phone via a text message. Additionally or alternatively, risk assessment module 110 may send an email to an email account of patient 218 that includes personal report 129(c).

In addition, or as an alternative, to providing patient 218 with genetic risk assessment 124 (e.g., via a response section and/or a patient report), risk assessment module 110 may send genetic risk assessment 124 to clinic 214 and/or healthcare provider 216. In one example, risk assessment module 110 may use genetic risk assessment 124 to create a patient report 129(a), describing genetic risk assessment 124 for patient 218, to provide to healthcare provider 216 and/or clinic 214. Risk assessment module may send patient report 129(a) to a variety of devices associated with healthcare provider 216 and/or clinic 214. In one embodiment, risk assessment module 110 may send patient report 129(a) to electronic health records system 202 associated with clinic 214 in a searchable structured data format.

Patient report 129(a) may include a variety of information. For example, patient report 129(a) may indicate whether genetic testing guidelines recommend testing patient 218 for genetic markers indicative of cancer. Additionally or alternatively, patient report 129(a) may indicate whether patient 218 has expressed (e.g., in one of responses 128) an interest in being tested for genetic markers indicative of cancer. Additionally or alternatively, patient report 129(a) may indicate whether the health insurance of patient 218 may cover the costs of testing patient 218 for genetic markers indicative of cancer.

In some examples, patient report 129(a) may also include a recommendation. For example, patient report 129(a) may include a recommendation that healthcare provider 216 discuss the patient's family history, genetic testing and/or genetic counseling with patient 218 during the patient's scheduled appointment. This recommendation may be based, at least in part, on a determination that genetic testing guidelines recommend testing patient 218 for genetic markers indicative of cancer. In one example, patient report 129(a) may include additional personal information pertaining to patient 218 to assist healthcare provider 216 in having a productive discussion during the patient's scheduled appointment about genetic screening. For example, patient report 129(a) may include demographic information for patient 218, family history information for patient 218, a current diagnosis of patient 218, a previous diagnosis of patient 218, and/or resource information for patient 218.

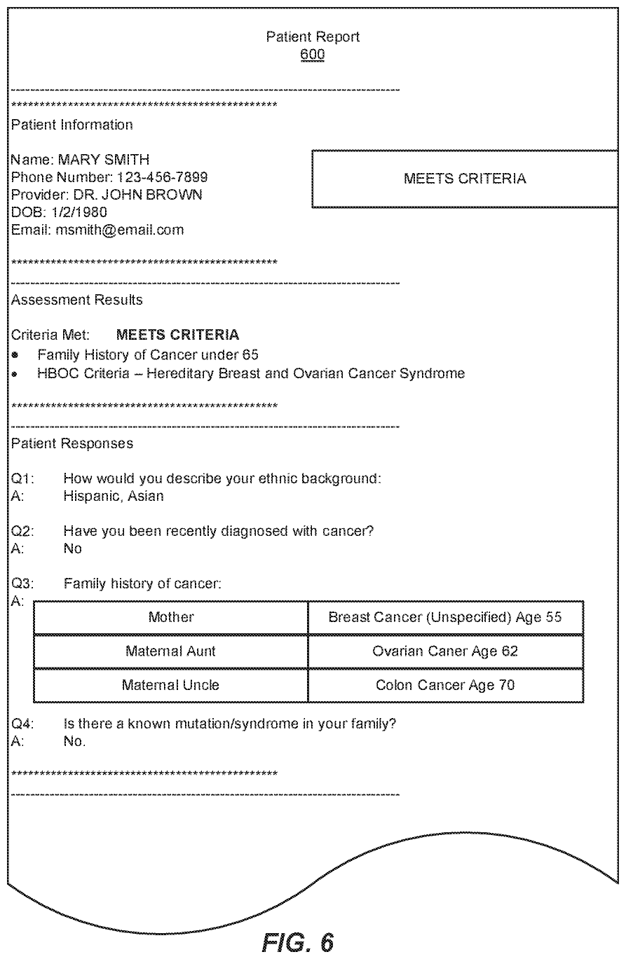

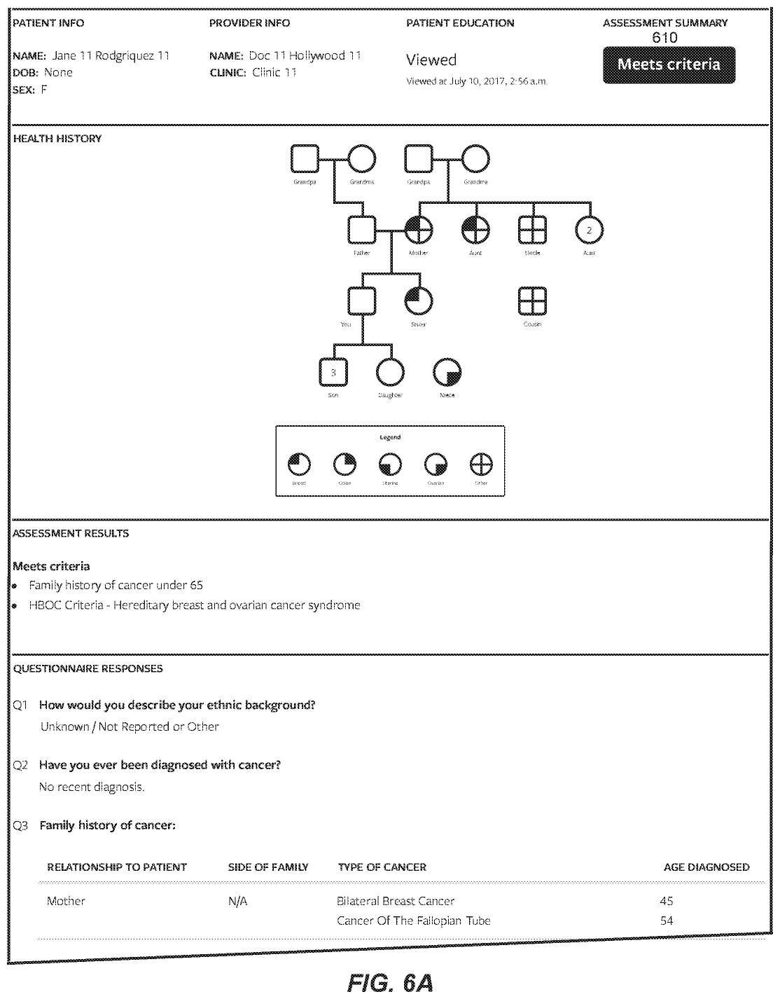

FIG. 6 illustrates a specific example of a patient report 600 about a patient. As shown in FIG. 6, patient report 600 includes patient information (e.g., the patient's name, date of birth, and email), assessment results (meets criteria), reasons for assessment results (family history of cancer under 65, HBOC criteria—hereditary breast and ovarian cancer), and the patient's responses submitted to electronic screening form 127.

Risk assessment module 110 may send patient report 129(a) automatically in response to a variety of triggers. In one example, risk assessment module 110 may send patient report 129(a) automatically in response to receiving responses 128 from patient 218. In other examples, risk assessment module 110 may send patient report 129(a), along with reports for other patients (e.g., as part of aggregate report 129(b)), on a periodic basis, as will be described in greater detail below.

In addition, or as an alternative, to creating patient report 129(a) for healthcare provider 216 and/or clinic 214, risk assessment module 110 may generate aggregate report 129(b) that includes and/or is based on genetic risk assessments for multiple patients. For example, aggregate report 129(b) may include risk assessment information for patients who have scheduled appointments with healthcare provider 216 and/or clinic 214. In one example, aggregate report 129(b) may include genetic risk assessment information for patients who have scheduled appointments within a certain timeframe. For example, the aggregate report may include genetic risk information for patients who have scheduled appointments within the upcoming day, week, and/or month.

Aggregate report 129(b) may include a variety of information. In one example, aggregate report 129(b) may include statistics for patients with scheduled appointments within the certain timeframe (e.g., within the upcoming week). For example, aggregate report 129(b) may include a number of patients (e.g., with appointments within the certain timeframe) who (i) received a digital request to fill out an electronic family history form or resource information form, (ii) completed an electronic family history form or resource information form, (iii) meet criteria for being tested for genetic markers indicative of cancer, (iv) have an elevated risk for inherited cancer, and/or (v) have a population risk for inherited cancer. Additionally or alternatively, aggregate report 129(b) may include a number of patients (e.g., who have scheduled appointments within the certain timeframe) who have already been tested for genetic markers indicative of cancer and/or a number of patients who have already been tested for genetic markers indicative of cancer and have received positive results.

In some examples, aggregate report 129(b) may keep track of various information for the patient population cared for by healthcare provider 216 and/or clinic 214. For example, aggregate report 129(b) may track patients who have been tested for genetic markers indicative of cancer. Similarly, aggregate report 129(b) may track patients who have been offered genetic counseling and/or genetic testing.

In some examples, aggregate report 129(b) may single out a subset of the patients who have scheduled appointments within the certain timeframe. For example, aggregate report 129(b) may single out a subset of the patients who genetic testing guidelines recommend receive genetic testing. Similarly, the aggregate report may single out a subset of the patients who have a higher genetic risk than other patients with scheduled appointments within the certain timeframe. In these examples, aggregate report 129(b) may also include a recommendation that a member of clinic 214 (e.g., healthcare provider 216) discuss genetic testing and/or genetic counseling with the subset of patients. For example, aggregate report 129(b) may include a list of patients (i.e., the subset of patients) whom healthcare provider 216 should prioritize for discussing health history, genetic testing, and/or genetic counseling during his or her upcoming patient appointments.

FIG. 6A is a block diagram of an example three-generation patient report 610, in accordance with one embodiment of the present disclosure. As shown in FIG. 6A, patient report 610 includes patient information (e.g., the patient's name, date of birth, and sex), assessment results (meets criteria), reasons for assessment results (family history of cancer under 65, HBOC criteria—hereditary breast and ovarian cancer), and the patient's responses submitted to electronic screening form 127.

Risk assessment module 110 may send patient report 129(a) automatically in response to a variety of triggers. In one example, risk assessment module 110 may send patient report 129(a) automatically in response to receiving responses 128 from patient 218. In other examples, risk assessment module 110 may send patient report 129(a), along with reports for other patients (e.g., as part of aggregate report 129(b)), on a periodic basis, as will be described in greater detail below.

In addition, or as an alternative, to creating patient report 129(a) for healthcare provider 216 and/or clinic 214, risk assessment module 110 may generate aggregate report 129(b) that includes and/or is based on genetic risk assessments for multiple patients. For example, aggregate report 129(b) may include risk assessment information for patients who have scheduled appointments with healthcare provider 216 and/or clinic 214. In one example, aggregate report 129(b) may include genetic risk assessment information for patients who have scheduled appointments within a certain timeframe. For example, the aggregate report may include genetic risk information for patients who have scheduled appointments within the upcoming day, week, and/or month.

Aggregate report 129(b) may include a variety of information. In one example, aggregate report 129(b) may include statistics for patients with scheduled appointments within the certain timeframe (e.g., within the upcoming week). For example, aggregate report 129(b) may include a number of patients (e.g., with appointments within the certain timeframe) who (i) received a digital request to fill out an electronic family history form or resource information form, (ii) completed an electronic family history form or resource information form, (iii) meet criteria for being tested for genetic markers indicative of cancer, (iv) have an elevated risk for inherited cancer, and/or (v) have a population risk for inherited cancer. Additionally or alternatively, aggregate report 129(b) may include a number of patients (e.g., who have scheduled appointments within the certain timeframe) who have already been tested for genetic markers indicative of cancer and/or a number of patients who have already been tested for genetic markers indicative of cancer and have received positive results.

In some examples, aggregate report 129(b) may keep track of various information for the patient population cared for by healthcare provider 216 and/or clinic 214. For example, aggregate report 129(b) may track patients who have been tested for genetic markers indicative of cancer. Similarly, aggregate report 129(b) may track patients who have been offered genetic counseling and/or genetic testing.

In some examples, aggregate report 129(b) may single out a subset of the patients who have scheduled appointments within the certain timeframe. For example, aggregate report 129(b) may single out a subset of the patients who genetic testing guidelines recommend receive genetic testing. Similarly, the aggregate report may single out a subset of the patients who have a higher genetic risk than other patients with scheduled appointments within the certain timeframe. In these examples, aggregate report 129(b) may also include a recommendation that a member of clinic 214 (e.g., healthcare provider 216) discuss genetic testing and/or genetic counseling with the subset of patients. For example, aggregate report 129(b) may include a list of patients (i.e., the subset of patients) whom healthcare provider 216 should prioritize for discussing health history, genetic testing, and/or genetic counseling during his or her upcoming patient appointments.

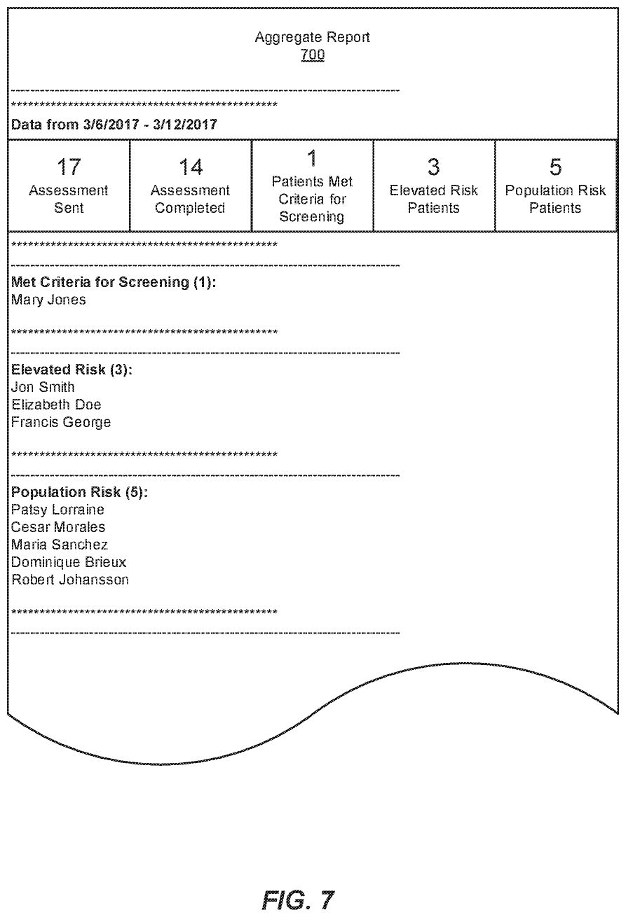

FIG. 7 illustrates a specific example of an aggregate report 700. As shown in FIG. 7, aggregate report 700 includes a date range covered by aggregate report 700 (i.e., Mar. 6, 2017 through Mar. 12, 2017), a number of assessments sent (i.e., 17), a number of assessments completed (i.e., 14), a number of patients who met criteria for screening (i.e., 1), a number of patients with an elevated risk (i.e., 3), a number of patients with a population risk (i.e., 5), and a list of names of patients who meet criteria for screening, have an elevated risk, or have a population risk.

As discussed above in connection with patient report 129(a), risk assessment module 110 may send aggregate report 129(b) to a variety of devices and/or entities. In some examples, risk assessment module 110 may send the aggregate report to healthcare provider 216 and/or to clinic 214 (e.g., by sending aggregate report 129(b) to electronic health records system 202 associated with clinic 214). In this example, risk assessment module 110 may send aggregate report 129(b) in a searchable structured data format.

Risk assessment module 110 may generate and/or send aggregate report 129(b) in response to a variety of events. In one embodiment, risk assessment module 110 may generate and/or send aggregate report 129(b) in response to receiving a request for an aggregate report. In another embodiment, risk assessment module 110 may periodically generate aggregate report 129(b), each generated instance of aggregate report 129(b) being generated in connection with a successive time period. In this embodiment, risk assessment module 110 may automatically send aggregate report 129(b) (e.g., to healthcare provider 216 and/or to clinic 214) periodically. To give a specific example, risk assessment module 110 may generate an instance of aggregate report 129(b) once a week that includes risk assessment information for patients scheduled to be seen by healthcare provider 216 and/or clinic 214 during the upcoming week.

In some examples, risk assessment module 110 may use information collected from responses 128 of patient 218 (and/or may aggregate information collected from the responses of multiple patients including patient 218) to identify characteristics of populations that are at risk for hereditary cancer. This type of analysis may be facilitated by storing the information gathered from electronic forms in a structured data format.

As discussed above, the disclosed systems and methods may improve the standard of care provided by healthcare providers by facilitating the consistent capture and assessment of health history using direct multiplatform patient engagement. By automating the collection of family history information and/or resource information, the disclosed systems and methods may facilitate identifying patients who meet genetic screening criteria for focused follow-up, reduce administrative burden, and gain population visibility. As described above, the disclosed systems and methods may receive a bulk patient data secure file transfer from a device associated with a clinic that identifies patients with upcoming appointments at the clinic. The disclosed systems and methods may use this information to provide patients who have upcoming appointments with a text message or an email requesting that the patient follow a link to complete a family history form and/or resource information form electronically. The disclosed systems and methods may use the family medical history, obtained from the responses provided by the patient to the family history form, to create a risk assessment. The disclosed systems and methods may further use the resource, obtained from the responses provided by the patient to the resource information form, to create a resource assessment. These assessments (and/or a report based on the assessments) may be transmitted to a patient device (e.g., via text or email), a provider device (e.g., via fax), and/or an electronic health records system associated with the provider (e.g., via a secure file transfer).

FIG. 8 is a block diagram of an example computing system 810 capable of implementing one or more of the embodiments described and/or illustrated herein. For example, all or a portion of computing system 810 may perform and/or be a means for performing, either alone or in combination with other elements, one or more of the steps described herein (such as one or more of the steps illustrated in FIG. 3). All or a portion of computing system 810 may also perform and/or be a means for performing any other steps, methods, or processes described and/or illustrated herein.

Computing system 810 broadly represents any single or multi-processor computing device or system capable of executing computer-readable instructions. Examples of computing system 810 include, without limitation, workstations, laptops, client-side terminals, servers, distributed computing systems, handheld devices, or any other computing system or device. In its most basic configuration, computing system 810 may include at least one processor 814 and a system memory 816.

Processor 814 generally represents any type or form of physical processing unit (e.g., a hardware-implemented central processing unit) capable of processing data or interpreting and executing instructions. In certain embodiments, processor 814 may receive instructions from a software application or module. These instructions may cause processor 814 to perform the functions of one or more of the example embodiments described and/or illustrated herein.

System memory 816 generally represents any type or form of volatile or nonvolatile storage device or medium capable of storing data and/or other computer-readable instructions. Examples of system memory 816 include, without limitation, Random Access Memory (RAM), Read Only Memory (ROM), flash memory, or any other suitable memory device. Although not required, in certain embodiments computing system 810 may include both a volatile memory unit (such as, for example, system memory 816) and a non-volatile storage device (such as, for example, primary storage device 832, as described in detail below). In one example, one or more of modules 102 from FIG. 1 may be loaded into system memory 816.

In some examples, system memory 816 may store and/or load an operating system 824 for execution by processor 814. In one example, operating system 824 may include and/or represent software that manages computer hardware and software resources and/or provides common services to computer programs and/or applications on computing system 810. Examples of operating system 824 include, without limitation, LINUX, JUNOS, MICROSOFT WINDOWS, WINDOWS MOBILE, MAC OS, APPLE'S IOS, UNIX, GOOGLE CHROME OS, GOOGLE'S ANDROID, SOLARIS, variations of one or more of the same, and/or any other suitable operating system.

In certain embodiments, example computing system 810 may also include one or more components or elements in addition to processor 814 and system memory 816. For example, as illustrated in FIG. 8, computing system 810 may include a memory controller 818, an Input/Output (I/O) controller 820, and a communication interface 822, each of which may be interconnected via a communication infrastructure 812. Communication infrastructure 812 generally represents any type or form of infrastructure capable of facilitating communication between one or more components of a computing device. Examples of communication infrastructure 812 include, without limitation, a communication bus (such as an Industry Standard Architecture (ISA), Peripheral Component Interconnect (PCI), PCI Express (PCIe), or similar bus) and a network.

Memory controller 818 generally represents any type or form of device capable of handling memory or data or controlling communication between one or more components of computing system 810. For example, in certain embodiments memory controller 818 may control communication between processor 814, system memory 816, and I/O controller 820 via communication infrastructure 812.

I/O controller 820 generally represents any type or form of module capable of coordinating and/or controlling the input and output functions of a computing device. For example, in certain embodiments I/O controller 820 may control or facilitate transfer of data between one or more elements of computing system 810, such as processor 814, system memory 816, communication interface 822, display adapter 826, input interface 830, and storage interface 834.

As illustrated in FIG. 8, computing system 810 may also include at least one display device 824 coupled to I/O controller 820 via a display adapter 826. Display device 824 generally represents any type or form of device capable of visually displaying information forwarded by display adapter 826. Similarly, display adapter 826 generally represents any type or form of device configured to forward graphics, text, and other data from communication infrastructure 812 (or from a frame buffer, as known in the art) for display on display device 824.

As illustrated in FIG. 8, example computing system 810 may also include at least one input device 828 coupled to I/O controller 820 via an input interface 830. Input device 828 generally represents any type or form of input device capable of providing input, either computer or human generated, to example computing system 810. Examples of input device 828 include, without limitation, a keyboard, a pointing device, a speech recognition device, variations or combinations of one or more of the same, and/or any other input device.

Additionally or alternatively, example computing system 810 may include additional I/O devices. For example, example computing system 810 may include I/O device 836. In this example, I/O device 836 may include and/or represent a user interface that facilitates human interaction with computing system 810. Examples of I/O device 836 include, without limitation, a computer mouse, a keyboard, a monitor, a printer, a modem, a camera, a scanner, a microphone, a touchscreen device, variations or combinations of one or more of the same, and/or any other I/O device.

Communication interface 822 broadly represents any type or form of communication device or adapter capable of facilitating communication between example computing system 810 and one or more additional devices. For example, in certain embodiments communication interface 822 may facilitate communication between computing system 810 and a private or public network including additional computing systems. Examples of communication interface 822 include, without limitation, a wired network interface (such as a network interface card), a wireless network interface (such as a wireless network interface card), a modem, and any other suitable interface. In at least one embodiment, communication interface 822 may provide a direct connection to a remote server via a direct link to a network, such as the Internet. Communication interface 822 may also indirectly provide such a connection through, for example, a local area network (such as an Ethernet network), a personal area network, a telephone or cable network, a cellular telephone connection, a satellite data connection, or any other suitable connection.

In certain embodiments, communication interface 822 may also represent a host adapter configured to facilitate communication between computing system 810 and one or more additional network or storage devices via an external bus or communications channel. Examples of host adapters include, without limitation, Small Computer System Interface (SCSI) host adapters, Universal Serial Bus (USB) host adapters, Institute of Electrical and Electronics Engineers (IEEE) 1394 host adapters, Advanced Technology Attachment (ATA), Parallel ATA (PATA), Serial ATA (SATA), and External SATA (eSATA) host adapters, Fibre Channel interface adapters, Ethernet adapters, or the like. Communication interface 822 may also allow computing system 810 to engage in distributed or remote computing. For example, communication interface 822 may receive instructions from a remote device or send instructions to a remote device for execution.

In some examples, system memory 816 may store and/or load a network communication program 838 for execution by processor 814. In one example, network communication program 838 may include and/or represent software that enables computing system 810 to establish a network connection 842 with another computing system (not illustrated in FIG. 8) and/or communicate with the other computing system by way of communication interface 822. In this example, network communication program 838 may direct the flow of outgoing traffic that is sent to the other computing system via network connection 842. Additionally or alternatively, network communication program 838 may direct the processing of incoming traffic that is received from the other computing system via network connection 842 in connection with processor 814.

Although not illustrated in this way in FIG. 8, network communication program 838 may alternatively be stored and/or loaded in communication interface 822. For example, network communication program 838 may include and/or represent at least a portion of software and/or firmware that is executed by a processor and/or Application Specific Integrated Circuit (ASIC) incorporated in communication interface 822.

As illustrated in FIG. 8, example computing system 810 may also include a primary storage device 832 and a backup storage device 833 coupled to communication infrastructure 812 via a storage interface 834. Storage devices 832 and 833 generally represent any type or form of storage device or medium capable of storing data and/or other computer readable instructions. For example, storage devices 832 and 833 may be a magnetic disk drive (e.g., a so-called hard drive), a solid state drive, a floppy disk drive, a magnetic tape drive, an optical disk drive, a flash drive, or the like. Storage interface 834 generally represents any type or form of interface or device for transferring data between storage devices 832 and 833 and other components of computing system 810. In one example, the elements included in additional elements 120 from FIG. 1 may be stored and/or loaded in primary storage device 832.

In certain embodiments, storage devices 832 and 833 may be configured to read from and/or write to a removable storage unit configured to store computer software, data, or other computer-readable information. Examples of suitable removable storage units include, without limitation, a floppy disk, a magnetic tape, an optical disk, a flash memory device, or the like. Storage devices 832 and 833 may also include other similar structures or devices for allowing computer software, data, or other computer-readable instructions to be loaded into computing system 810. For example, storage devices 832 and 833 may be configured to read and write software, data, or other computer-readable information. Storage devices 832 and 833 may also be a part of computing system 810 or may be a separate device accessed through other interface systems.

Many other devices or subsystems may be connected to computing system 810. Conversely, all of the components and devices illustrated in FIG. 8 need not be present to practice the embodiments described and/or illustrated herein. The devices and subsystems referenced above may also be interconnected in different ways from that shown in FIG. 8. Computing system 810 may also employ any number of software, firmware, and/or hardware configurations. For example, one or more of the example embodiments disclosed herein may be encoded as a computer program (also referred to as computer software, software applications, computer-readable instructions, or computer control logic) on a computer readable medium. The term “computer-readable medium,” as used herein, generally refers to any form of device, carrier, or medium capable of storing or carrying computer-readable instructions. Examples of computer-readable media include, without limitation, transmission type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The computer-readable medium containing the computer program may be loaded into computing system 810. All or a portion of the computer program stored on the computer-readable medium may then be stored in system memory 816 and/or various portions of storage devices 832 and 833. When executed by processor 814, a computer program loaded into computing system 810 may cause processor 814 to perform and/or be a means for performing the functions of one or more of the example embodiments described and/or illustrated herein. Additionally or alternatively, one or more of the example embodiments described and/or illustrated herein may be implemented in firmware and/or hardware. For example, computing system 810 may be configured as an Application Specific Integrated Circuit (ASIC) adapted to implement one or more of the example embodiments disclosed herein.

FIG. 9 is a block diagram of an example network architecture 900 in which client systems 910, 920, and 930 and servers 940 and 945 may be coupled to a network 950. As detailed above, all or a portion of network architecture 900 may perform and/or be a means for performing, either alone or in combination with other elements, one or more of the steps disclosed herein (such as one or more of the steps illustrated in FIG. 3). All or a portion of network architecture 900 may also be used to perform and/or be a means for performing other steps and features set forth in the instant disclosure.

Client systems 910, 920, and 930 generally represent any type or form of computing device or system, such as example computing system 810 in FIG. 8. Similarly, servers 940 and 945 generally represent computing devices or systems, such as application servers or database servers, configured to provide various database services and/or run certain software applications. Network 950 generally represents any telecommunication or computer network including, for example, an intranet, a WAN, a LAN, a PAN, or the Internet. In one example, client systems 910, 920, and/or 930 and/or servers 940 and/or 945 may include all or a portion of system 100 from FIG. 1.

As illustrated in FIG. 9, one or more storage devices 960(1)-(N) may be directly attached to server 940. Similarly, one or more storage devices 970(1)-(N) may be directly attached to server 945. Storage devices 960(1)-(N) and storage devices 970(1)-(N) generally represent any type or form of storage device or medium capable of storing data and/or other computer-readable instructions. In certain embodiments, storage devices 960(1)-(N) and storage devices 970(1)-(N) may represent Network-Attached Storage (NAS) devices configured to communicate with servers 940 and 945 using various protocols, such as Network File System (NFS), Server Message Block (SMB), or Common Internet File System (CIFS).

Servers 940 and 945 may also be connected to a Storage Area Network (SAN) fabric 980. SAN fabric 980 generally represents any type or form of computer network or architecture capable of facilitating communication between a plurality of storage devices. SAN fabric 980 may facilitate communication between servers 940 and 945 and a plurality of storage devices 990(1)-(N) and/or an intelligent storage array 995. SAN fabric 980 may also facilitate, via network 950 and servers 940 and 945, communication between client systems 910, 920, and 930 and storage devices 990(1)-(N) and/or intelligent storage array 995 in such a manner that devices 990(1)-(N) and array 995 appear as locally attached devices to client systems 910, 920, and 930. As with storage devices 960(1)-(N) and storage devices 970(1)-(N), storage devices 990(1)-(N) and intelligent storage array 995 generally represent any type or form of storage device or medium capable of storing data and/or other computer-readable instructions.

In certain embodiments, and with reference to example computing system 810 of FIG. 8, a communication interface, such as communication interface 822 in FIG. 8, may be used to provide connectivity between each client system 910, 920, and 930 and network 950. Client systems 910, 920, and 930 may be able to access information on server 940 or 945 using, for example, a web browser or other client software. Such software may allow client systems 910, 920, and 930 to access data hosted by server 940, server 945, storage devices 960(1)-(N), storage devices 970(1)-(N), storage devices 990(1)-(N), or intelligent storage array 995. Although FIG. 9 depicts the use of a network (such as the Internet) for exchanging data, the embodiments described and/or illustrated herein are not limited to the Internet or any particular network-based environment.

In at least one embodiment, all or a portion of one or more of the example embodiments disclosed herein may be encoded as a computer program and loaded onto and executed by server 940, server 945, storage devices 960(1)-(N), storage devices 970(1)-(N), storage devices 990(1)-(N), intelligent storage array 995, or any combination thereof. All or a portion of one or more of the example embodiments disclosed herein may also be encoded as a computer program, stored in server 940, run by server 945, and distributed to client systems 910, 920, and 930 over network 950.

As detailed above, computing system 810 and/or one or more components of network architecture 900 may perform and/or be a means for performing, either alone or in combination with other elements, one or more steps of an example method for automatically generating genetic risk assessments.

While the foregoing disclosure sets forth various embodiments using specific block diagrams, flowcharts, and examples, each block diagram component, flowchart step, operation, and/or component described and/or illustrated herein may be implemented, individually and/or collectively, using a wide range of hardware, software, or firmware (or any combination thereof) configurations. In addition, any disclosure of components contained within other components should be considered example in nature since many other architectures can be implemented to achieve the same functionality.

In some examples, all or a portion of example system 100 in FIG. 1 may represent portions of a cloud-computing or network-based environment. Cloud-computing environments may provide various services and applications via the Internet. These cloud based services (e.g., software as a service, platform as a service, infrastructure as a service, etc.) may be accessible through a web browser or other remote interface. Various functions described herein may be provided through a remote desktop environment or any other cloud based computing environment.

In various embodiments, all or a portion of example system 100 in FIG. 1 may facilitate multi-tenancy within a cloud-based computing environment. In other words, the software modules described herein may configure a computing system (e.g., a server) to facilitate multi-tenancy for one or more of the functions described herein. For example, one or more of the software modules described herein may program a server to enable two or more clients (e.g., customers) to share an application that is running on the server. A server programmed in this manner may share an application, operating system, processing system, and/or storage system among multiple customers (i.e., tenants). One or more of the modules described herein may also partition data and/or configuration information of a multi-tenant application for each customer such that one customer cannot access data and/or configuration information of another customer.

According to various embodiments, all or a portion of example system 100 in FIG. 1 may be implemented within a virtual environment. For example, the modules and/or data described herein may reside and/or execute within a virtual machine. As used herein, the term “virtual machine” generally refers to any operating system environment that is abstracted from computing hardware by a virtual machine manager (e.g., a hypervisor). Additionally or alternatively, the modules and/or data described herein may reside and/or execute within a virtualization layer. As used herein, the term “virtualization layer” generally refers to any data layer and/or application layer that overlays and/or is abstracted from an operating system environment. A virtualization layer may be managed by a software virtualization solution (e.g., a file system filter) that presents the virtualization layer as though it were part of an underlying base operating system. For example, a software virtualization solution may redirect calls that are initially directed to locations within a base file system and/or registry to locations within a virtualization layer.

In some examples, all or a portion of example system 100 in FIG. 1 may represent portions of a mobile computing environment. Mobile computing environments may be implemented by a wide range of mobile computing devices, including mobile phones, tablet computers, e-book readers, personal digital assistants, wearable computing devices (e.g., computing devices with a head-mounted display, smartwatches, etc.), and the like. In some examples, mobile computing environments may have one or more distinct features, including, for example, reliance on battery power, presenting only one foreground application at any given time, remote management features, touchscreen features, location and movement data (e.g., provided by Global Positioning Systems (GPS), gyroscopes, accelerometers, etc.), restricted platforms that restrict modifications to system-level configurations and/or that limit the ability of third-party software to inspect the behavior of other applications, controls to restrict the installation of applications (e.g., to only originate from approved application stores), etc. Various functions described herein may be provided for a mobile computing environment and/or may interact with a mobile computing environment.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks.

These software modules may include script, batch, or other executable files that may be stored on a computer-readable storage medium or in a computing system. In some embodiments, these software modules may configure a computing system to perform one or more of the example embodiments disclosed herein.

In addition, one or more of the modules described herein may transform data, physical devices, and/or representations of physical devices from one form to another. For example, one or more of the modules recited herein may receive input from a patient to an electronic form and transform the input into a genetic risk assessment for the patient. Additionally or alternatively, one or more of the modules recited herein may transform a processor, volatile memory, non-volatile memory, and/or any other portion of a physical computing device from one form to another by executing on the computing device, storing data on the computing device, and/or otherwise interacting with the computing device.

The preceding description has been provided to enable others skilled in the art to best utilize various aspects of the example embodiments disclosed herein. This example description is not intended to be exhaustive or to be limited to any precise form disclosed. Many modifications and variations are possible without departing from the spirit and scope of the instant disclosure. The embodiments disclosed herein should be considered in all respects illustrative and not restrictive. Reference should be made to the appended claims and their equivalents in Determining the scope of the instant disclosure.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and have the same meaning as the word “comprising.”

1. A computer-implemented method for automatically generating genetic risk assessments, at least a portion of the method being performed by a computing device comprising at least one processor, the method comprising:
receiving a data share, derived from an electronic health records system, that indicates that a patient has scheduled an appointment with a healthcare provider;
in response to identifying that the patient has scheduled an appointment with the healthcare provider, sending a digital family history request to the patient in preparation for the scheduled appointment, the digital family history request comprising a link to a third-party portal and a request to access and complete an electronic family history form using the third-party portal;
receiving, through the third-party portal, responses to the family history form that the patient submitted using the third-party portal; and
generating a genetic risk assessment for the patient based on the responses received through the third-party portal.

2-6. (canceled)

7. The computer-implemented method of claim 1, further comprising:
identifying guidelines for testing for genetic markers indicative of cancer; and
determining, based on the patient's genetic risk assessment, whether the guidelines recommend testing the patient for the genetic markers indicative of cancer.

8. The computer-implemented method of claim 1, further comprising automatically sending a report comprising the patient's genetic risk assessment to at least one of the healthcare provider and a clinic associated with the healthcare provider.

9. The computer-implemented method of claim 8, wherein sending the report to at least one of the healthcare provider and the clinic comprises sending the report to at least one of the healthcare provider and the clinic by sending the report to the electronic health records system in a searchable structured data format.

10. (canceled)

11. The computer-implemented method of claim 8, wherein sending the report to at least one of the healthcare provider and the clinic comprises automatically sending the report to at least one of the healthcare provider and the clinic in response to receiving the responses from the patient.

12-13. (canceled)

14. The computer-implemented method of claim 8, wherein the report includes a recommendation that that the healthcare provider discuss at least one of genetic testing and genetic counseling with the patient during the patient's scheduled appointment, and the method further comprises including the recommendation in the report in response to determining, based on the patient's genetic risk assessment, that genetic testing guidelines recommend testing the patient for genetic markers indicative of cancer.

15. (canceled)

16. The computer-implemented method of claim 1, further comprising:
generating, based at least in part on the patient's genetic risk assessment, an aggregate report of a plurality of patients, including the patient, who have scheduled appointments with at least one of the healthcare provider and a clinic associated with the healthcare provider; and
automatically sending the aggregate report to at least one of the healthcare provider and the clinic.

17. The computer-implemented method of claim 16, wherein the aggregate report comprises at least one of:
a number of patients who received a digital request to fill out an electronic family history form;
a number of patients who completed an electronic family history form;
a number of patients who meet criteria for being tested for genetic markers indicative of cancer;
a number of patients with an elevated risk for inherited cancer; and
a number of patients with a population risk for inherited cancer.

18. The computer-implemented method of claim 16, wherein the aggregate report:
identifies a subset of patients from within the plurality of patients with a higher genetic risk than other patients within the plurality of patients; and
recommends that the healthcare provider discuss at least one of genetic testing and genetic counseling with the subset of patients.

19. The computer-implemented method of claim 16, wherein the aggregate report further comprises at least one of:
a number of patients from within the plurality of patients who have been tested for genetic markers indicative of cancer; and
a number of patients who have been tested for genetic markers indicative of cancer with positive results.

20. The computer-implemented method of claim 16, wherein the aggregate report tracks at least one of:
patients who have been tested for genetic markers indicative of cancer; and
patients who have been offered at least one of genetic counseling and genetic testing.

21. The computer-implemented method of claim 16, wherein:
generating the aggregate report comprises periodically generating the aggregate report, each generated instance of the aggregate report being generated in connection with a successive time period; and
automatically sending the aggregate report comprises periodically sending the aggregate report.

22-24. (canceled)

25. The computer-implemented method of claim 1, further comprising:
receiving responses to the electronic family history form from a plurality of additional patients;
aggregating information collected from the patient's responses and the responses collected from the plurality of additional patients; and
analyzing the aggregated information to identify characteristics of populations that are at risk for hereditary cancer.

26. The computer-implemented method of claim 1, wherein the steps of the method are performed by a server managed by an entity that provides genetic screening services, wherein the electronic health records system automatically transmits the data share to the server managed by the entity in response to determining that the patient has been scheduled for the appointment.

27-29. (canceled)

30. The computer-implemented method of claim 1, further comprising providing the patient with a personal report comprising at least one of:
the patient's genetic risk assessment;
the patient's responses to the family history form; and
educational information,
wherein providing the patient with the personal report comprises at least one of:
sending, to the patient, a link to the personal report; and
sending, to the patient, an email that includes the personal report.

31. (canceled)

32. A system for automatically generating genetic risk assessments, the system comprising:
a server managed by an entity that provides genetic testing services;
a data share module, stored in memory of the server, that receives, from an electronic health records system associated with a health clinic, a data share that indicates that a patient has scheduled an appointment with a healthcare provider at the health clinic;
a patient engagement module, stored in the memory, that, in response to identifying that the patient has scheduled an appointment with the healthcare provider, sends a digital family history request to the patient in preparation for the scheduled appointment, the digital family history request comprising:
a link to a third-party portal; and
a request to access and complete an electronic family history form using the third-party portal;
a response module, stored in the memory, that receives responses to the family history form that the patient submitted using the third-party portal;
a risk assessment module, stored in the memory, that generates a genetic risk assessment for the patient based on the responses received through the third-party portal; and
at least one physical processor configured to execute the receiving module, the patient engagement module, the collection module, and the risk assessment module.

33. A non-transitory computer-readable medium comprising one or more computer-executable instructions that, when executed by at least one physical processor of a computing device, cause the computing device to:
receive a data share, derived from an electronic health records system, that indicates that a patient has scheduled an appointment with a healthcare provider;
in response to identifying that the patient has scheduled an appointment with the healthcare provider, send a digital family history request to the patient in preparation for the scheduled appointment, the digital family history request comprising:
a link to a third-party portal; and
a request to access and complete an electronic family history form using the third party portal;
receive, through the third-party portal, responses to the family history form that the patient submitted using the third-party portal; and
generate a genetic risk assessment for the patient based on the responses received through the third-party portal.

34. (canceled)

35. The computer-implemented method of claim 1, further comprising:
generating, based at least in part on the genetic risk assessments, a three-generation patient pedigree, wherein the three-generation patient pedigree comprises determining at least one of:
number of aunts/uncles related to the patient;
number of brothers/sisters related to the patient; and
number of sons/daughters related to the patient.

36. (canceled)

37. The system for automatically generating genetic risk assessments of claim 32, the system further comprising:
a patient pedigree module, stored in the memory, that generates a three-generation patient pedigree based on the genetic risk assessment for the patient, wherein the three-generation patient pedigree comprises determining at least one of:
number of aunts/uncles related to the patient;
number of brothers/sisters related to the patient; and
number of sons/daughters related to the patient.

38. (canceled)

39. The non-transitory computer-readable medium comprising one or more computer-executable instructions of claim 33, further causing the computing device to:
generate a three-generation patient pedigree module, stored in the memory, based on the genetic risk assessment for the patient, wherein the three-generation patient pedigree comprises determining at least one of:
number of aunts/uncles related to the patient;
number of brothers/sisters related to the patient; and
number of sons/daughters related to the patient.

40-42. (canceled)
